(19) United States
(12) Patent Application Publication
(10) Pub. No.: US 2021/0269373 A1
(43) Pub. Date: Sep. 2, 2021

(54) SOIL RESTORATION SYSTEM

(71) Applicant: PROFILE PRODUCTS L.L.C., Buffalo Grove, IL (US)

(72) Inventors: Kevin Scott SPITTLE, Vero Beach, FL (US); Michael Dan ROBESON, Fort Collins, CO (US); Stephen Robert MACSHANE, Huntley, IL (US)

(73) Assignee: PROFILE PRODUCTS L.L.C., Buffalo Grove, IL (US)

(21) Appl. No.: 17/255,498

(22) PCT Filed: Jul. 1, 2019
(86) PCT No.: PCT/US2019/040062

§ 371 Date: Dec. 23, 2020

Related U.S. Application Data
(60) Provisional application No. 62/691,686, filed on Jun. 29, 2018.

(51) Int. Cl. C05F 11/08; C05D 9/02; C05F 11/10; C05G 1/00; C05G 5/30; C05G 5/40; C12N 1/20; A01C 21/00

(52) U.S. Cl. CPC C05F 11/08; C05D 9/02; C05F 11/10; A01C 21/00; C05G 5/30; C05G 5/40; C12N 1/20; C05G 1/00

(57) ABSTRACT

A soil restoration system including a plurality of dry particles, each particle inoculated with dehumidified biological material including at least one species of cyanobacteria, the cyanobacteria being physically supported by the particles, and the cyanobacteria being activatable by a threshold amount of moisture so that when the cyanobacteria is activated at an application site, the cyanobacteria multiplies to form a biological soil crust capable of facilitating plant seed catchment and vascular plant germination at the application site.

### TECHNICAL FIELD

This application claims the benefit of U.S. provisional application Ser. No. 62/691.686 filed Jun. 29, 2018, the disclosure of which is hereby incorporated in its entirety by reference herein.

The present disclosure is related to an inoculant and an inoculate particle designed to assist with soil restoration in arid and semi-arid as well as mote humid areas and methods of producing and using the same.

### BACKGROUND

Lack of precipitation, high evaporation rates, and other factors related to climate and weather factors have provided challenging conditions for vegetation growth in certain areas of the Earth designated as arid and semi-arid. As a result, the arid and semi-arid areas are susceptible to increased erosion and desertification. Naturally-occurring biological soil crusts, communities of living organisms on the soil surface of the arid and semi-arid areas, protect the soil. Yet, the biological soil crusts are relatively fragile and take a long time to recover when disturbed. Various soil restoration attempts have been made to preserve and revive the biological soil crusts. For example, one method includes salvaging a biological crust from one area and reapplying it at the remediation site. Yet, this method is time-consuming and unsustainable as it disturbs the original site. Thus, a need exists to develop a more reliable, sustainable, and environmentally friendly soil restoration method.

### SUMMARY OF THE INVENTION

The presently disclosed soil restoration system includes porous ceramic particles inoculated with one or more genus of cyanobacteria, optionally combined with additional components. The cyanobacteria is prepared offsite in a laboratory or bioreactor. The additional components may be nutrients and/or additional organisms such as fungi spores, lichen, bryophytes, green algae, or a combination thereof. The inoculated particle may be disseminated onto a remedial site such that when a sufficient moisture activates the dormant organisms of the inoculant, the organisms start to grow and multiply to form a biological soil crust capable of facilitating plant seed catchment and vascular plant germination.

In another embodiment, the soil restoration system includes porous ceramic particles inoculated with fungi and designed for soil restoration system in areas more humid than semi-arid areas. The inoculated particles may be dried and applied onto a remedial site, where moisture activates the dormant fungi.

In at least one embodiment, a soil restoration system is disclosed. The system includes a plurality of dry particles, each particle inoculated with dehumidified biological material including at least one species of cyanobacteria, the cyanobacteria being physically supported by the particles, and the cyanobacteria being activatable by a threshold amount of moisture so that when the cyanobacteria is activated at an application site, the cyanobacteria multiplies to form a biological soil crust capable of facilitating plant seed catchment and/or vascular plant germination at the application site. The particles may be ceramic particles. The cyanobacteria may include Microcoleus genera, Nostoc genera, of their combination. The system may also include at least one of a cyanobacteria food source, macronutrients, micronutrients, tackifier, bio stimulants, or plant hormones. The biological material may further include at least one species of fungi. The particle total porosity may be about 50 to 95 volume %. Application density of the plurality of particles on the application site may be about 500 to 2000 lbs/acre. The particles may have a concentration of the dehumidified biological material of about 500 to 40,000 g/acre of particles.

In an additional embodiment, a soil restoration system is disclosed. The system includes a plurality of dry particles, each particle inoculated with dehumidified biological material including at least one species of cyanobacteria, the cyanobacteria being physically supported by the particles, and the cyanobacteria being activatable by a threshold amount of moisture so that when the cyanobacteria is activated at an application site, the cyanobacteria multiplies to form a biological soil crust capable of facilitating plain seed catchment and/or vascular plant germination at the application site. The system also includes growing medium or mulch having a density of about 60 kg/m³ or lower. The plurality of particles and the growing medium may form a mixture. Alternatively or in addition. the growing medium may form a protective layer over the plurality of particles. The system may also include at least one of a cyanobacteria food source, macronutrients, micronutrients, tackifier, bio stimulants, or plant hormones. The biological portion may further include at least one species of fungi. The biological portion may include bacterial genera matching a separate biological material to be present at the application site. The application density of die plurality of particles on the application site may be about 500 to 2000 lbs/acre.

In yet another embodiment, a soil restoration system may include a liquid inoculant including at least one species of cyanobacteria to be applied at an application site such that when the cyanobacteria is activated at an application site, the cyanobacteria multiplies to form a biological soil crust capable of facilitating plant seed catchment and vascular plant germination at the application site. The system also includes growing medium or mulch having a density of about 60 kg/m³ or lower. The liquid inoculant and the growing medium may form a mixture. Alternatively, or additionally, the growing medium may form a protective layer over the plurality of particles. The system may also include at least one of a cyanobacteria food source, macronutrients. micronutrients, tackifier, bio stimulants, or plant hormones. The application density of the liquid inoculant on die application site may be about 5 to 20 gal/acre.

### BRIEF DESCRIPTION OF THE DRAWINGS

FIG. 1 depicts a view of a non-limiting example of a biological soil crust in an arid or semi-arid area:

FIG. 2 is a photograph of Microcoleus-coated particles disclosed herein serving as supports for cyanobacteria growth in various media;

FIG. 3 is a photograph of a non-limiting example of an untreated ceramic particle according to one or more embodiments;

FIG. 4 is a photograph of an example particle coated with an example inoculant;

FIG. 5 is a photograph of an example coated particle with cyanobacteria fiber attached to the particle;

FIG. 6 shows Examples 1-40 of treated awl untreated soil samples of the First Trial;

FIGS. 7 and 8 show randomized Examples 1-40;

FIG. 9 shows Chlorophyll a test results of Examples 1-40 of the First Trial;

FIG. 10 depicts Examples 41-80 of treated and untreated soil samples of the Second Trial;

FIG. 11 shows Chlorophyll a test results of Examples 41-80 of the Second Trial;

FIG. 12 is a photograph of an example plot with an application of a liquid cyanobacteria treatment and a mulch cap;

FIG. 13 is a photograph of example particles coated with cyanobacteria;

FIG. 14 is a photograph of an example plot with an application of dry inoculated particles of FIG. 13; and

FIG. 15 shows pigment results for Examples 81-90 three and six months, respectively, after installation of the Third Trial.

### DETAILED DESCRIPTION

Embodiments of the present disclosure are described herein. It is to be understood, however, that the disclosed embodiments are merely examples and other embodiments may take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the present disclosure. As those of ordinary skill in the art will understand, various features illustrated and described with reference to any one of the figures may be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Except where expressly indicated, all numerical quantities in this description indicating dimensions or material properties are to be understood as modified by the word “about” in describing the broadest scope of the present disclosure.

The first definition of an acronym or other abbreviation applies to all subsequent uses herein of the same abbreviation and applies mutatis mutandis to normal grammatical variations of the initially defined abbreviation. Unless expressly stated to the contrary, measurement of a property is determined by the same technique as previously or later referenced for the same property.

The description of a group or class of materials as suitable tor a given purpose in connection with one or more embodiments implies that mixtures of any two or more of the members of the group or class are suitable. Description of constituents in chemical terms refers to the constituents at the time of addition to any combination specified in the description, and does not necessarily preclude chemical interactions among constituents of the mixture once mixed. The first definition of an acronym or other abbreviation applies to all subsequent uses herein of the same abbreviation and applies mutatis mutandis to normal grammatical variations of the initially defined abbreviation. Unless expressly slated to the contrary, measurement of a property is determined by tire same technique as previously or later referenced for the same property.

Arid and semi-arid areas cover almost one-third of the total land area of the world. The arid zones are usually divided into hyper-arid, arid, and semi-arid, depending on the amount of precipitation these zones receive. Aridity results from the presence of dry, descending air. Thus, the location of the arid regions correlates with anticyclonic conditions such as in the regions lying under the anticyclones. Arid conditions arc also associated with the “rain shadow” of the mountain ranges which disrupt the structures of cyclones providing moisture.

Among common characteristics of any arid zone is excessive heat, wide variation in temperatures, and inadequate, variable precipitation only marginally capable of supporting vegetation growth. Other factors may worsen the situation and growth conditions such as unsustainable agricultural practices and other human-related activities. As a result, arid zone soils are very vulnerable to both wind and water erosion. Soil fixation and conservation are thus important, and vegetation plays a crucial role in the conservation efforts.

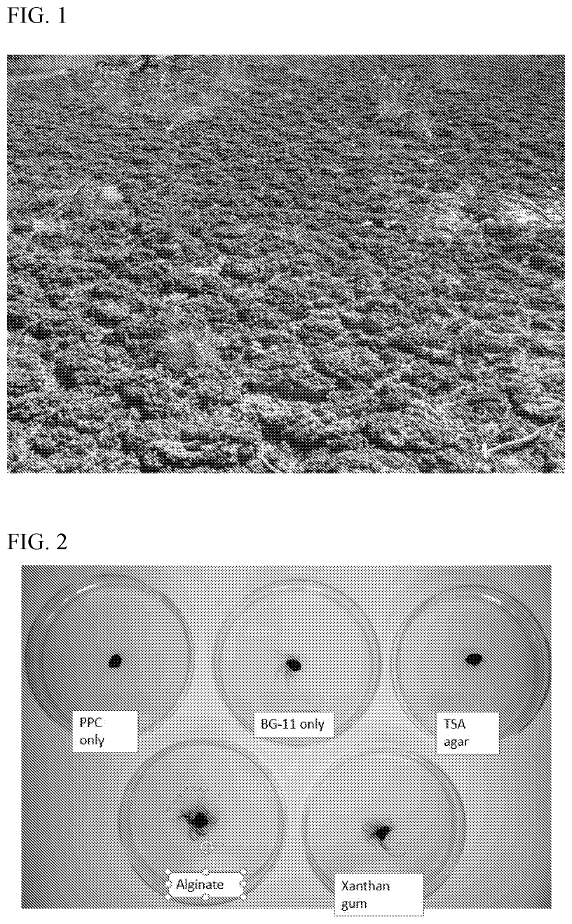

Yet, the vegetation in the arid and semi-arid areas is more susceptible to disturbance than vegetation in other climate zones as seeds are less likely to catch onto the soil surface and germination is more difficult due to low precipitation. But vegetation may be supported on biological soil crusts which present communities of living organisms on the soil surface in the arid and semi-arid zones. The organisms may include cyanobacteria, lichens, fungi, bryophytes, algae, and so on, which symbiotically coexist in the upper-most layers of the soil surface and form a biological basis for the formation of the soil crusts. Without these organisms, the arid and semi-arid soils would not be capable to effectively support vegetation. The biological soil crusts typically form in the open spaces between vascular plants. The single-cell organisms such as cyanobacteria or fungi spores are capable of colonizing bare soil first. Higher organisms such as mosses can follow the colonization once the single-cell organisms stabilized the soil. The biological soil crust may form a layer of several mm to several cm high, depending on the conditions and types of organisms present. A non-limiting example of a biological crust is illustrated in FIG. 1.

The biological soil crusts arc also known as cryptogamic, microbiotic, microphytic. or cryptobiotic soils. While the herein-described biological soil crusts can be found on neatly any type of soil, their ability to grow is usually limited by presence of other plant types and competition for light. Thus, the arid and semi-arid areas with limited vascular plant vegetation provide growth conditions with less competition. Yet, especially with respect to fungi, their ability to grow in humid areas may surpass ability of other organisms named above as at least some fungi thrive in humid conditions with less light.

These biological soil crusts assist with carbon fixation, nitrogen fixation, soil stabilization, reduction of dust emissions, positively influence water retention and soil albedo. In addition, the biological soil crusts can encourage seed catchment, increase germination, and boost nutrient levels in vascular plants.

The biological soil crusts have several advantages compared to vascular plants in the conditions of the arid and semi-arid zones. For example, the biological soil crust organisms are capable of growing vegetatively. Unlike seeds, the organisms do not typically die when there is lack of precipitation as the organisms may go dormant. The organisms also typically tolerate tougher soils, high pH, high salinity, temperature swings, high amount of UV, and lower amount of precipitation than vascular plants. If stored properly, the organisms may retain viability for decades.

But due to the overall arid zone environment, even biological soil crusts are quite susceptible to disturbance. When damaged, long time periods are required for the biological soil crust to recover and fulfill the beneficial functions. Specific conditions of the region, availability of precipitation, frequency and strength of wind and water erosion, continued presence or absence of additional disturbances determine the length of recovery. For example, recovery of the biological soil crust is generally faster in a fine-textured soil and moister environments and slower in coarser soils and dry environments. Yet, even the speedier recovery can take years or decades.

Natural disasters such as flooding, mudslides, earthquakes, and other conditions such as excessive grazing, construction, mining, recreation activities, etc. or conditions determined by microclimates, may render soils in any region lifeless or more susceptible to deterioration such that the soils need to be remediated. The remediation efforts are thus not limited to the arid and semi-arid areas, but encompass regions characterized by a higher degree of humidity, for example Mediterranean, tropical dry, highlands, cool summer regions, transition regions, etc. in tropical, temperate/mesothermal, or continental/microthermal climate zones.

Several methods have been developed to encourage regrowth of the biological soil crusts, where the crust was disturbed, damaged, or removed. A typical method, salvaging, utilizes a biological soil crust removed from one site, which is scraped and reapplied at the remedial site. But this method requires removal of the biological soil crust from a location, which may become vulnerable or which may be handicapped as a reason of this remedial method. Thus, unless the disturbance of the original site is purposeful and presents a planned disturbance, the practice is unsustainable.

An alternative method is to apply one or more components of the biological soil crusts developed in an artificial environment such as a greenhouse or lab, onto the remedial site to start regrowth. The organisms may be applied in a form of inoculated substrate material. For example, the substrate material may be a flat fibrous stripe and the inoculant may be attached to the substrate via an adhesive. The substrate used may be natural or synthetic fiber. Yet, the fiber may present an obstacle for the organisms and natural fiber may disintegrate. An alternative substrate may be in a form of pellets, which has shown to be non-economical if applied on a larger scale. Other substrates have been proposed, but present a challenge for various reasons. For example, a significant impediment associated with developing of the biological soil crusts appears to be in growing the cyanobacteria and then transferring the cyanobacteria to a material/carrier for application into the field. For instance, most substrates cannot hold sufficient quantities of water for a sufficient length of time, do not significantly reduce erosive effects, do not permanently improve the soil, or a combination thereof. Additionally, green house scale growth of the biological soil crusts on a 2-dimensional sand mix is not an economically viable method.

Thus, there remains a need for an inoculant for soil remediation in at least the arid and semi-arid areas, or alternatively in humid areas, which is easily applicable, capable of supporting the inoculated organisms, which does not burden the environment of the remedial site, and, among other properties, resists water and wind erosion.

In one or more embodiments of the present disclosure, an inoculated particle is disclosed. The inoculated particle may be applied to any type of soil in any climate zone. The inoculated particle is especially suitable as a soil remediation article in arid and semi-arid areas. The inoculated particles disclosed herein solve one or more drawbacks described above.

An arid region is any region having a severe lack of available water with annual rainfall between 0 to about 300 mm. A semi-arid region is a region with annual precipitation between 200 and 800 mm. Soil remediation of regions having more than 800 mm annual precipitation using the inoculated particles disclosed herein is also contemplated.

The inoculated particle includes an inoculant or inoculum. The inoculant may include a biological portion or biological material. The biological portion or material may include one or more types of organisms naturally occurring in the biological soil crusts of the area to be remediated. For example, the organisms may include one or more types of cyanobacteria. Example cyanobacteria or cyanophyte include any bacteria classified under cyanobacteria. For example, the cyanobacteria may include classes Chroobacteria, Hormogoneae, and Gloeobacteria, orders Chroococcales, Gloeobacterales, Nostocales, Oscillatoriales, Pleurocapsales, and Stigonematales, families Prochloraceae and Prochlorotrichaceae, and the genera Halospirulina, Planktotricoides, Prochloron, Prochlorothrix, and Rubidibacter. Specific genera which may be part of the inoculant may include Nostoc and Microcoleus. Yet, additional genera of cyanobacteria may be included, for example Aphanocapsa, Aphanothece, Chamaesiphan, Chondrocystis, Chroococcus, Chroogloeocystis, Coefosphaerium, Crocosphaera, Cyanobacterium, Cyanobium, Cyanodictyon, Cyanosarcina, Cyanothece, Dactylococcopsis, Geminocystis, Gloeocapsa, Gloeothece, Halothececluster: Euhalothece, Halptece, Johannesbaptistia, Merismopedia, Microcystis, Radiocystis, Rhabdoderma, Rubidibacter, Snowella, Sphaerocavum, Synechococcus, Synechocystis, Thermosynechococcus, Woronichinia from the order Chroococcales; Gloeobacter from the order Gleobacterales; Coleodesmium, Fremyella, Hassallia, Microchaete, Petalonema, Rexia, Spirirestis, Tolypothrix, Anabaena, Anabaenopsis, Aphanizomenon, Aulosira, Cyanospira, Cylindrospermopsis, Cylindrospermum, Mojavia, Nodularia, Nostoc, Raphidiopsis, Richelia, Trichormus, Calothrix, Gloeotrichia, Rivularia, Brasilonema, Scytonema, Scytonematopsis from the order Nostocales; Arthronema, Arthrospira, Blennothrix, Crinalium, Geitlerinema, Halo-micronema, Halospirulina, Hydrocoleum, Jaaginema, Katagnymene, Komvophoron, Leptolyngbya, Limnothrix, Lyngbya, Microcoleus, Osciliatoria, Phormidium, Planktolyngbya, Planktothricoides, Plankiothrix, Plectonema, Pseudanabaena, Pseudophormidium, Schizothrix, Spirulina, Starria, Symploca, Trichocoleus, Trichodesmium, Tychonema from the order Oscillatoriales; Chroococcidiopsis, Dermocarpa, Dermocarpella, Myxosarcina, Pleurocapsa, Solentia, Stanieria, Xenococcus, Prochloron, Prochlorococcus, Prochlorothrix from the order Pleurocapsales; Capsosira, Chlorogloeopsis, Fischerella; Hapalosiphon, Mastigocladopsis, Mastigocladus, Nostochopsis, Sligonema, Symphyonema, Symphyonemopsis, Umezakia, Westiellopsis from the order Stigonematales.

A combination of two or more genera named above is contemplated. As was stated above, the inoculate may contain two or more different genera such as, for example. Microcoleus and Nostoc. The ratio of one genera to the other genera may be 1:1, 1:2, 1:3, 1:4, 1:5, 1:6, 1:7, 1:8, 1:9, 1:10, 2:3, 4:5, or a different ratio.

The biological portion or material may include one or more types of algae. Example algae may include green algae Phylum Chlorophyta: diatoms—classes Diatomophyceae or Bacillariophyceae, yellow-green algae or Xanthophyceae, and eukaryotic algae Eustigmatophyceae.

In addition or alternatively, the biological portion or material may contain one or more types of lichen. Lichens represent composite organisms of algae or cyanobacteria living among filaments of fungi in a symbiotic relationship. The lichen may include phycolichens, associations of fungi and eukaryotic green algae, capable of sustaining on water vapor. The lichen morphologies may include squamulous (corn flake-like with the thallus margin upraised, Clavicidium lacinulatum, Placidium squamulosum, Psora decipiens), foliose (intricate leafy thallus, Physconia sp., Xanthoparmelia sp.), fruticose (morphologically complex thallus such as podetia of Cladonia) or crustose (Sarcogyne mitziae, Acaraspora schleicheri). The lichen may include cyanolichen, an association of fungi and cyanobacteria. Morphologies may include gelatinous such as Collema, crustose (Peltula spp., Heppia spp.), or both.

The biological portion or material may likewise include one or more types of bryophyte. Bryophyte represents non-vascular land plants including mosses and liverworts, which require the highest amount of moisture from the named organisms associated with the biological crusts. Thus, bryophyte may be more suitable for humid applications. Bryophyte may include moss species such as Bryum spp., Syntrichia spp., Crossidium spp., Pterygoneurum spp., liverwort species such as Riccia.

Alternatively, or in addition to the organisms named above, the biological portion or material may also include one or more types of fungi. The fungi may include species which may colonize tissues of lichens (lichenicolous), mosses (bryophilous), and/or grasses of biocrusts in the arid and semi-arid areas or more moist environments. Such fungi may include Hymenoascomycetes (Chaetominum sp.); Loculoascomycetes (Mycosphaerella sp., Graphyllium sp. such as Graphyllium permundum, Phaeospora sp. such as Pleospora richtophensis, Leptosphaeria sp., Preussia sp., Kalmusia utahensis, Macroventuria wentii), Lecanorales (Poelcinula/Melanospora sp.); Basidiomycetes (Cyphellostereum sp.); Anamorphic/mitosporic species(Acremonium spp., Alternaria sp. such as Alternaria tenuissima, Ascochyta sp., Aspergillus spp. such as Aspergillus leporis, Aspergillus ustus, Aureobasidium sp. such as Aureobasidium pullans, Bipolaris sp., Chrysosporium/Geomyces pannorus, Cladosporium spp. such as Cladosparium herbarum, Cladosporium macrocarpum, Embellisia spp, such as Embellisia chlamydospora, Embellisia tellustris, Epicoccum sp. such as Epicoccum purpurascens, Fusarium spp. such as Fusarium equiseti, Fusarium flocciferum, Heteroconium sp., Monodictys sp. such as Monodictys putredinis, Mortierella sp. such as Mortierella alpina, Myrothecium sp., Papulaspora sp., Phoma spp. such as Phoma anserine, Phoma fimeti, Phoma leveillei, Phoma nebulosa, Pseudozyma sp., Sclerococcum sp., Stachybotrys sp. such as Stachybotrys cf. cylindrospor, Taeniolella sp., Trichoderma sp., Ulocladium spp. such as Ulocladium chartarum, Ulocladium cf. multiform); and Mycelia sterilia (Mycelium sterile dematiaceum spp.). Other fungi species are contemplated.

Other species of any of the above-named organisms are contemplated. The species of choice may be determined based on a number of factors such as cost, survival rate, availability, whether the species are native to the remedial site, etc. The biological portion or material may include genera and species matching a separate biological portion or material of the remedial or application site. Alternatively, the biological portion may contain genera and species which differ from those found at the application site.

Besides the biological portion or material, the inoculate may include additional components for the benefit of the cyanobacteria and/or other organisms of the biological portion. In addition, some of the components may serve other functions. For example, at least some of the carbohydrates may include sugars whose sticky consistency may aid in soil aggregation. Such sugars may be sugars which are normally secreted by one or more types of organisms outside of their cell for this purpose.

The additional components may include a growth promoting nutrient medium. The medium may contain biological nutrients: carbohydrates such as polysaccharides including starch, dextrin, glycogen, galactomannas or gums such as guar gum, beta-mannan, carob, fenugreek, tara gum, konjac gum, gum acacia (arabic), karaya, tragacanth, arabinoxylan (soluble), gellan, xanthan, seaweed polysaccharides such as agar-agar, alginate, carrageenan; soil plant macronutrients: P, N, Ca, Mg, S, Mg; soil/plant micronutrients: Mn, Fe, B, Zn, Co, Mb, Cl; bio stimulants such as humic acid: plant hormones; a combination thereof, or the like.

The inoculant's biological portion may be grown in a liquid bath, bioreactor, or a tank containing a liquid culture. The biological portion may be grown indoors or outdoors. Various bacterial, cyanobacterial genera, fungi, and/or other organisms forming the biological portion may be grown together or separately. The liquid bath may contain one or more of the components named above supporting growth and multiplication.

Alternatively, the biological portion, for example the cyanobacteria, may be grown in a humid, but not liquid, environment. Another viable example may be phycolichens, sustainable on water vapor. The growth may thus take place directly within and/or on the particle with or without direct exposure to liquid water bath.

When the liquid bath reaches a desired concentration of the biological portion, the organisms may be dried, freeze dried, dehumidified, or otherwise rendered dormant, but remaining viable upon activation. For example, the biological portion may be dried to about 0.5, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49, 50, or more weight or volume % of the liquid bath. The biological portion may be combined with the additional components named above to form the inoculant. The combining may be done while the biological portion is still moist. Alternatively, the biological portion and the additional components may be combined once the biological portion is dehumidified. The combining may be performed while the biological portion is or is not dormant. For example, a part of the biological portion may be dormant while the remainder may be non-dormant. Either part may be about 1, 2, 3, 4, 5, 6, 7, 8, 9, 10, 15, 20, 25, 30, 35, 40, 45, 50, 55, 60, 65, 70, 76, 80, 85, 90, 95 or more %.

The inoculant may be prepared in a dry or liquid form. The inoculant may be then applied to a particle which provides housing to the inoculant. For example, individual particles may be soaked in a liquid inoculant. The particles may be submerged in the inoculant and serve as a physical support for the growing organisms. An example of herein-disclosed particles placed into various inoculants/media containing and supporting growth of the biological portion can be seen in FIG. 2. The photograph was taken 6 days after plating. The upper left Petri dish contains a Microcoleus-coated particle with no liquid broth, the middle upper photograph shows a Petri dish including a Microcoleus-coated particle in BG-11 medium, the right upper Petri dish contains a Microcoleus-coated particle in TSA agar. The bottom left Petri dish includes a Microcoleus-coated particle m alginate and the bottom right Petri dish includes a Microcoleuscoated particle in xantham gum.

In an alternative embodiment, individual particles may be soaked in the inoculant at a remedial site onto which the particles may be spread before the liquid inoculant is applied. In another embodiment, the liquid inoculant may be applied onto the native soil without the particles. For example, the liquid-only inoculant may be applied as a secondary or subsequent treatment after a first treatment which included both the particles and the inoculant.

After application of a liquid broth/inoculant at a remedial site, additional treatments may be installed such as a mulch or growing medium cap or a protective layer to prevent erosion, a supply of nutrients which are missing in the native soil such as P or other nutrients named herein, a bio stimulant such as seaweed extract or other bio stimulant named herein, a food source for the organisms such as guar or other food sources named herein, a tackifier for mulch fibers such as guar, the like, or a combination thereof. Alternatively still, the liquid inoculant may be applied together with die growing medium or mulch, for example after being mixed together in a hydroseeder tank. Alternatively or in addition, growing medium or mulch may be applied over the particles or liquid inoculant at the application site as a protective and/or supportive layer.

The mulch or growing medium may be a mulch or growing medium disclosed in U.S. Pat. No. 10,266.457 which is hereby incorporated by reference. The mulch composition or growing medium, and in particular, a fibrous mulch composition or growing medium, may include about 5 to about 95 weight % of tree bark mixed with about 95 to about 5 weight % of wood components, based on the total weight of the mulch composition or growing medium. The mulch composition or growing medium may include 100 weight % fibrous pine wood components. The mulch composition or growing medium may include about 10 weight % of tree bark and about 90 weight % of wood components, based on the total weight of the mulch composition or growing medium. The mulch composition or growing medium may include about 20 to about 70 weight % of tree bark and about 30 to about 80 weight % of wood components or vice versa, based on the total weight of the mulch composition or growing medium, the mulch composition or growing medium may include about 50 to about 60 weight % of tree bark and about 40 to about 50 weight % of wood components, based on the total weight of the mulch composition or growing medium. The mulch composition or growing medium may include about 90 weight % of tree bark and about 10 weight % of wood components, based on the total weight of the mulch composition or growing medium. Typically, about 1 to about 99, 90. 85, 80, 75, 70, 65, 60, 55, 50, 45, 40, 35, 30, 25, 20, 15, 10, 5 weight % of tree bark, such as pine, is combined with about 99, 95, 90, 85, 80, 75, 70, 65, 60, 55, 50, 45, 40, 35, 30, 25, 20, 15, 10, 5, 1 weight % of wood components, based on the total weight of the mulch composition or growing medium. Alternatively, about 1 to about 99, 90, 85, 80, 75, 70, 65, 60, 55, 50, 45, 40, 35, 30, 25, 20, 15, 10, 5 weight % of wood components may be combined with about 99, 95, 90, 85, 80, 75, 70, 65, 60, 55, 50, 45, 40, 35, 30, 25, 20, 15, 10, 5, 1 weight % of tree bark such as pine, based on the total weight of the mulch composition or growing medium.

The mulch composition or growing medium may further include about 0 to about 10 weight % or at least 1, 2, 3, 4, 5, 6, 7, 8, 9, 10, 12, 15, 17, 20, or more weight % of additional components, based on the total weight of the mulch composition or growing medium, as set forth below. Examples of such additional components include, but are not limited to, fertilizer(s). macronutrient(s), micronutrient(s), mineral(s), binder(s), natural gum(s), interlocking manmade fiber(s), and the like, and combinations thereof. In general, these additional components in total are present in an amount of less than about 20, 15, or 10 weight % of the total weight of the mulch composition or growing medium. More preferably, the additional components in total arc present in an amount from about 1 to about 15 weight % of the total weight of the mulch composition or growing medium. Additionally, soil way be present in an amount of about 20 weight % or less, about 15 weight % or less, or about 5 weight % or less of the total weight of the mulch composition or growing medium. The soil may be present in an amount of about 0.1 to about 20 weight % of the total weight of the mulch composition or growing medium. Soil may be native soil from the remediation site. Soil may also be absent from the mulch composition or growing medium.

The dry bulk density of the growing medium or mulch may be, in order of increasing preference, about 6 lb/ft³ (96.11 kg/m³) or less, 4 lb/ft³ (64.07 kg/m³) or less, 3 lb/ft³ (48.06 kg/m³) or less, or 2 lb/ft³ (32.04 kg/m³) or less. The dry bulk density of the mulch composition or growing medium may be about 1.5 lb/ft³ (24.03 kg/m³) to about 6 lb/ft³ (96.11 kg/m³), about 2 lb/ft³ (32.04 kg/m³) to about 4 lb/ft³ (64.07 kg/m³), about 2.2 lb/ft³ (35.24 kg/m³) to about 2.6 lb/ft³ (41.65 kg/m³). The wet bulk density of the mulch composition or growing medium may be, in order of increasing preference, about 15 lb/ft³ (240.28 kg/m³) or less, 10 lb/ft³ (160.18 kg/m³) or less, 8 lb/ft³ (128.15 kg/m³) or less, 6 lb/ft³ (96.11 kg/m³) or less, 4 lb/ft³ (64.07 kg/m³) or less, 3 lb/ft³ (48.06 kg/m³) or less, or 2 lb/ft³ (32.04 kg/m³) or less. The wet bulk density of the mulch composition or growing medium may be about 1 lb/ft³ (16.02 kg/m³) to about 20 lb/ft³ (320.37 kg/m³), about 2.2 lb/ft³ (35.24 kg/m³) to about 10 lb/ft³ (160.18 kg/m³), about 2.4 ft³ (38.44 kg/m³)to about 15 lb/ft³ (240.28 kg/m³), about 2.6 (41.65 kg/m³) to 10 lb/ft³ (160.18 kg/m³), about 2.8 lb/ft³ (44.85 kg/m³) to about 7 lb/ft³ (112.13 kg/m³), about 3.0 lb/ft³ (48.06 kg/m³) to about 5 lb/ft³ (80.09 kg/m³).

Compared to other substrates, growing media or mulches, the growing medium disclosed herein is processed in the pressurized vessel by a process described below resulting in fiber which is thinner and longer, which has higher surface area ratio, much lower density, as well as smaller median fiber diameter. Due to the growing medium preparation process described herein, the lignin with the growing medium components melts and the resultant fiber is shaped differently compared to other media. For example, coir particles are generally spherical in shape with a smaller aspect ratio than the growing medium fiber. Bark particles and perlite are generally cylindrical. Peat, PTS, and WTS particles arc more elongated than coir, bark, and perlite, but remain wider and shorter than growing medium fiber.

The growing medium disclosed herein is prepared by mixing wood and/or bark components together, optionally with additional components named above, to form an initial mixture in step a). In step b), the initial composition is heated to an elevated temperature to kill microbes in a pressurized vessel. Typically, the heating step may be conducted at a temperature in the range of about 250° F. (121° C.) or lower to about 500° F. (260° C.) or higher, about 300° F. (149° C.) to about 400° F. (204° C.), about 320° F. (160° C.) to 380° F. (about 193° C.). The heating step may be conducted for a time sufficient to kill microbes. The heating step may result in a substantially sterile mulch composition or growing medium such that the mulch composition or growing medium is free from bacteria or other living organisms. In step c), the initial composition is processed through a refiner to form the mulch composition or growing medium. The refiner is usually operated at a lower temperature than the temperature used in step b). The refiner may be operated at a temperature in the range of about 70° F. (21° C.) to about 400° F. (204° C.), about 150° F. (66° C.) to about 350° F. (176° C.), about 200° F. (93° C.) to about 300° F. (148° C.). In step d). the mulch composition or growing medium is dried at temperatures of about 400° F. (204° C.) to about 600° F. (316° C.) for the time sufficient to reduce the moisture content of the mulch composition or growing medium to a value less than about 45 w eight %, less than about 25 weight %, or less than about 15 weight %, based on the total weight of the mulch composition or growing medium. Example equipment for drying of the mulch composition or growing medium in step d) may be a Hash tube dryer capable of drying large volumes of mulch composition or growing medium in a relatively short length of time due to the homogeneous suspension of the particles inside the flash tube dryer. While suspended in the heated gas stream, maximum surface exposure is achieved, giving the growing medium uniform moisture. The moisture content of the mulch composition or growing medium may be from about 10 to about 50 weight %, about 20 to about 40 weight %, about 25 to about 35 weight % of the total weight of the mulch composition or growing medium 20. In an optional step e), the mulch composition or growing medium is further refined, and the additional components set forth above may be added.

TABLE 1
Substrate particle distribution in substrates
and growing medium containing 100 vol./wt. %
of one type of substrate or growing medium.

| Type of Substrate | | Growing | Peat | Perlite | Bark |
|---|---|---|---|---|---|
| [100 vol. %] | | medium | Particle | Particle | Particle |
| Sieves | Particle | Particle | Dis- | Dis- | Dis- |
| [Mesh/ | Range | Distribution | tribution | tribution | tribution |
| μm] | [nm] | [%] | [%] | [%] | [%] |
| ¼” | >6.3 | 0.3 | 8.9 | 0.0 | 25.2 |
| 6300 | | | | | |
| #4/ | 4.75-6.2 | 0.1 | 6.1 | 2.0 | 9.6 |
| 4750 | | | | | |
| #8/ | 2.36-4.74 | 12.4 | 17.8 | 52.8 | 28.1 |
| 2360 | | | | | |
| #16/ | 1.18-2.35 | 23.8 | 18.1 | 23.9 | 16.7 |
| 1180 | | | | | |
| #25/ | 0.71-1.17 | 24.2 | 20.1 | 8.8 | 11.0 |
| 710 | | | | | |
| #50/ | 0.3-0.7 | 71.5 | 20.2 | 11.8 | 9.2 |
| 300 | | | | | |
| #100/ | 0.15-0.29 | 10.3 | 6.9 | 0.8 | 0.2 |
| 150 | | | | | |
| Pan/ | <0.15 | 7.3 | 1.9 | 0.0 | 0.0 |
| <150 | | | | | |

The growing medium may thus have elongated, relatively thin fibers with greater length to width ratio than other substrates, as can be seen in Table 2, such that the growing medium has higher available water. Additionally, the elongated fiber of the growing medium provides reinforcement to a substrate the growing medium is applied to or over and promotes organism and/or plant growth in a faster manner.

TABLE 2
Average length to width ratio of particles in sieves #16 and
#50 of various substrates and of the growing medium.

| | Sieve #16/180 μm | Sieve #50/300 μm |
|---|---|---|
| Type of | 1.8-2.36 mm Particle Range | 0.30-0.71 mm Particle Range |
| Substrate | Average length to | Average length to |
| [100 | Width Ratio Range | Width Ratio Range |

| vol. %] | Lower | Higher | Lower | Higher |
|---|---|---|---|---|
| GM | 14.899:1 | 30.602:1 | 39.615:1 | 55.507:1 |
| Peat | 1.463:1 | 2.010:1 | 3.498:1 | 6.323:1 |
| Perlite | 1.070:1 | 1.133:1 | 1:1 | 1.260:1 |
| Bark | 1.255:1 | 1.520:1 | 1.702:1 | 2.019:1 |
| Coir | 1.720:1 | 1.840:1 | 1.051:1 | 1.349:1 |
| PTS | 7.260:1 | 7.392:1 | 2.543:1 | 14.497:1 |
| WTS | 1.805:1 | 4.368:1 | 4.942:1 | 13.329:1 |

In at least about one embodiment, about 10 to 80 weight % of the growing medium has fiber with an average length to width ratio, also referred to as an aspect ratio, of 14:1 to 31:1. Alternatively, at least about 40 to 50 weight % of the growing medium has fiber with an average length to width ratio of 14:1 to 31:1. In at least one embodiment, about 10 to 80 weight % of the growing medium has fiber with an average length to width ratio of 39:1 to 56:1. In an alternative embodiment, about 20 to 70 weight % of the growing medium has fiber with an average length to width ratio of 39:1 to 56:1. Alternatively, about 40 to 50 weight % of the growing medium has fiber with an average length to width ratio of 39:1 to 56:1.

At least about 1, 2, 5, 10, 15, 20, 25. 30, 35, 40, 45, 50, 55, 60, 65, 70, 75, 80, 85, 90 weight or volume % of the growing medium fibers have an average length to width aspect ratio equal to or greater than 8:1, 9:1, 10:1, 14:1, 15:1, 18:1, 20:1, 22:1, 25:1, 18:1, 30:1 in sieve #16. About 10 to 80, 20 to 70, 30, to 60,40 to 50 weight or volume % of the growing medium fiber has the aspect ratio of equal to or greater than 8:1, 9:1, 10:1, 14:1, 15:1, 18:1, 20:1, 22;1, 25:1, 18:1, 30:1 in sieve #16.

At least about 1, 2, 5, 10, 15, 20, 25, 30, 35, 40, 45, 50, 55, 60, 65, 70, 75, 80, 85, 90 weight or volume % of the growing medium fibers have an average length to width aspect ratio equal to or greater than 16:1, 18:1, 20:1, 22:1, 24:1, 26:1, 28:1, 30:1, 32:1, 33:1, 34:1, 36:1, 38:1, 39:1, 40:1, 42:1, 44:1, 46:1, 48:1, 50:1, 52:1, 55:1 in sieve #50. About 10 to 80, 20 to 70, 30 to 60, 40 to 50 weight or volume % of the growing medium fiber has the aspect ratio of equal to or greater than 16:1, 18:1, 20:1, 22:1, 24:1, 26:1, 28:1, 30:1, 32:1, 33:1, 34:1, 36:1, 38:1, 39:1, 40:1, 42:1, 44:1, 46:1, 48:1, 50:1. 52:1, 55:1 in sieve #50.

At least about 1, 2, 3, 4, 5, 6, 7, 8, 9, 10, 15, 20, 25, 30, 35, 40, 45, 50, 55, 60, 65, 70, 75, 80, 85, 90, 95 weight or volume % of the fibers, based on the total weight of the fibers, have an average length to width ratio of about 8:1 to 35:1, 10:1 to 30:1, 12:1 to 28:1, 15:1 to 25:1, 18:1 to 23:1, 20:1 to 22:1 in sieve #16, and/or 15:1 to 60:1, 20:1 to 55:1, 25:1 to 50:1, 28:1 to 45:1, 25:1:40:1. 28:1 to 38:1, 30:1 to 35:1 sieve #50. Alternatively, at least about 1 to 90, 10 to 80, 20 to 70, 30 to 60, 40 to 50 weight or volume % of the fibers, based on the total weight of the fibers, have an average length to width ratio of at about 8:1 to 35:1, 10:1 to 30:1, 12:1 to 28:1, 15:1 to 25:1, 18:1 to 23:1, 20:1 to 22:1 in sieve #16, and/or 15:1 to 60:1, 20:1 to 55:1, 25:1 to 50:1, 28:1 to 45:1, 25:1; 40:1, 28:1 to 38:1, 30:1 to 35:1 in sieve #50.

The herein-disclosed particle may be coated with the inoculant on the inside, on the outside, or both. Any coating method is contemplated including spraying, painting, soaking, dipping, or the like to apply the inoculant onto the particles. The method needs to consider fragility of the organisms such that the application does not destroy the biological portion. For example, diluting and blending the biological portion for easier application should be made with caution as lowering viscosity of the inoculant containing the biological portion may induce damage to the organisms.

The inoculant may be applied onto the entire surface of the particle or a portion of the surface. The surface portion onto which the inoculant is applied may be about 1, 2, 3, 4, 5, 6, 7, 8, 9, 10, 15, 20, 25, 30, 35, 40, 45, 50, 55, 60, 65, 70, 76, 80, 85, 90, 95 or more %. The inoculum may be applied on the inside of the particle such that the inoculant tills a certain percentage of the particle cavities. For example, the filled cavities may represent about 1, 2, 3, 4, 5, 6, 7, 8, 9, 10, 15, 20, 25, 30, 35, 40, 45, 50, 55, 60, 65, 70, 76, 80, 85, 90, 95 or more % of the particle cavities. The inoculant may lie applied in the particle, on the particle, or both while the biological portion is or is not dormant or active.

With respect to the growth and application of the organisms in or on the particle, certain types of species may be grown and applied onto the particle in a different manner. For example, while cyanobacteria or fungi may be grown inside the particle, lichens may be applied on top of the particle. In an alternative non-limiting example, one or more genus of the cyanobacteria may be grown in the particle while one or more different genus of the cyanobacteria may be grown in a liquid reactor and coated or applied on top of the particle.

In one or more embodiments, the organisms may be grown in and/or in the particle, as described herein. Once grown, the particles with the dormant organisms may be harvested, while a moisture content is kept at a level lower than a moisture content required to activate the organisms, the threshold moisture level or content. The particles may be then transported and/or used in dry applications.

The inoculated particles may then be treated and/or packaged. Example treatment may include adding and/or removing texture to/from at least a portion the inoculated particles. All or at least a portion of the inoculated particles may be smooth, consistent, regular, even, uniform, raised, rough, inconsistent, irregular, uneven, coarse, or a combination thereof. The treatment may include applying additional substances such as a polysaccharide coating, pesticide, insecticide, drying, moisturizing, freezing, thawing, or a combination thereof. The biological portion may be rendered dormant before, during, and/or after the treatment and/or packaging.

The amount of the inoculant may be predetermined and automatically metered. As a result, each particle may have a uniform weight. Alternatively, particles containing various amounts of inoculant may be prepared such that a soil treatment having particles of various weights are prepared.

An example amount of the inoculant applied to a single particle may be about 0.01 to 20 weight %, 0.05 to 15 weight %, or 0.1 to 10 weight %, based on the weight of a single particle. The amount of the inoculant applied to a single particle may be about 0.01, 0.5, 0.1, 1,5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11, 11.5, 12, 12.5, 13, 13.5, 14, 14.5, 15, 15.5, 16, 16.5, 17, 17.5, 18, 18.5, 19, 19.5, or 20 weight %, based on the weight of a single particle.

The particle housing the inoculant may serve a variety of purposes. The particle provides a means of transportation or a delivery vehicle of the inoculant to the remedial site. Additionally, the particle provides protect km such that the inoculant remains at the site until the right conditions occur for the organisms to activate such as after precipitation or when moisture is artificially applied, and the dormant organisms start to develop.

The particles may be porous particles having pores, channels, chambers, conduits, ducts, spaces, voids, openings, or the like throughout. The particles may have a total porosity of at least about 30%, 35%, 40%, 45%, 50%, 55%, 60%, 65%, 70%, 75%, or more. At least 20%, 30%, 40%, 50%, 60%, or 70% of the total porosity may be capillary porosity or porosity relating to pores capable of holding water by capillary forces. The particle's total porosity may be about 50 to 95 volume %, 60 to 90 volume %, or 65 to 85 volume %. The particle's total porosity may be about 50, 55, 60, 65, 70, 75, 80, 85, 90, 95, or more volume 5.

Due to the porosity, the inoculant may become embedded within individual pores of the particle, inside the particle, on the surface of the particle, or a combination thereof. Higher moisture content of the particle assists with penetration of the particle with the inoculant. For example, the particle may have a moisture content of about 0 to 5%, 1 to 4%, or 2 to 3%. For example, the particle may have a moisture content of about 0.1, 0.2, 0.3, 0.4, 0.5, 0.6, 0.7, 0.8, 0.9, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, or 5.0%. The particle moisture content should not exceed moisture content which keeps the organisms such as the cyanobacteria dormant. In other words, the particle should not have a moisture content sufficient to activate the dormant organisms.

An example density of the particles before inoculation may be about 30-45 lbs/ft³ (480-720 kg/m³), 32-40 lbs/ft³ (512-640 kg/m³), or 35-37 lbs/ft³ (560-592 kg/m³). The density of the inoculated particles may increase by about 1 to 25%, 3 to 20%, or 6-14% when compared to the particles' state before inoculation. The density of the inoculated particles may increase by about 1, 2, 3, 4, 5, 6, 7, 8, 9, 10, 11, 12, 13, 14, 15, 16, 17, 18, 19, 20, 21, 22, 23, 24, or 25% when compared to the particles' state before inoculation.

Alternatively, the inoculant may be prepared to enable formation of a film or coating on the surface of the porous particle without penetrating all the pores such that at least some, or all, of the particle pores remain inoculant-free. Alternatively still, the coating may be designed in such a way that an increased amount of moisture will react with the coating and at least partially dissolve the coating. Subsequently, the inoculant may penetrate the particle pores post-application. This may be advantageous in certain types of applications, for example in an area with a possible wafer runoff. An application as a film enables proper metering of the amount of die inoculant on each particle. The inoculant may have better access to moisture on die surface of the particle, but may penetrate within the pores upon contact with moisture such that the inoculant is not washed away.

The particles may be ceramic particles. The particles may be prepared by calcining a clay. Example clays include smectite clays, for example smectite clays containing montmorillinite an/or opal CT (cristobalite. tridymite). Other clays such as bentonite, beidellite, nontronite, hectorite, saponite, attapugite, sepiolite may also be suitable. The ceramic particles may have better resistance to chemical and mechanical erosion than prior art substrates. Additionally, the ceramic particles may impart better erosion resistance once incorporated into a matrix.

The clay may contain about 5-60%, 10-50%, or 20-35%, or montmorillonite and about 40-90%, 30-80%, or 45-60% opal CT, respectively, with varying content of quartz, other clays, minerals, and impurities, as measured by x-ray diffraction. The clay may include about 25-30% montmorillonite, about 2-7% quartz, and about 50-60% opal CT. The calcination process may result in modification of at least some of the materials into minerals. A non-limiting example of a calcined particle may include about 1-2% montmorillonite, about 20-30% illite, about 2-5% quartz, and about 40-50% opal CT, as measured by X-ray diffraction.

The calcining process results in particles having a variety of sizes. Additional processes such as screening or sieving may be implemented to arrive at desirable particles sizes. The particles suitable for the disclosed application should fulfill several requirements. For example, the particles should have a sufficient size and appropriate shape to accommodate a coating layer of the inoculant, serve as a physical support for the growing organisms within the inoculant, and/or absorbing a desirable amount of the inoculant within its pores, indentations, or cavities.

The shape of the particle may be regular or irregular. The particle may have a substantially circular or oval cross-section. The cross-section may be regular or irregular, symmetrical or asymmetrical.

Other types of particles are contemplated, natural as well as synthetic. Example particles may include sand, gravel, a polymer, or the like. Porosity of the particles may be the same as above or lower. The particles may be non-porous such that a coating of the inoculant is applied strictly onto the surface without penetration into the particle. A mixture of various particles is also contemplated. For example, less than 100% of particles may be calcined clay. In a non-limiting example, a batch of particles may contain 50-99%, 60-95%, or 70-90% ceramic porous particles, the balance being particles including sand, grave, polymer, or a combination thereof.

Additionally, the particle has to be large and heavy enough to remain at the remedial site despite wind and water erosion from rainfall as well as surface runoff. Thus, different remedial sites may require different size and/or weight of the particles to be effectively spread on and kept at the site.

An example particle size may be less than about 0.60 mm (0.0236 inch), 0.60-0.85 mm (0.0236-0.0335 inch), 0.85-2 mm (0.0335-0.0787 inch), or larger than about 2 mm (0.0787 inch). Example particles may range from about 20 μm (0.00079 inch) to 12,700 μm (0.5 inch), 100 μm (0.003937 inch) to 10,000 μm (0.39370 inch), or 2,000 μm (0.07874 inch) to 5,000 μm (0.19685 inch). The example particles may have a sieve size of about 600 mesh to ½ inch mesh, 170 mesh to 18 mesh, or 10 mesh to 4 mesh. Any size within the ranges named above is contemplated. Larger and smaller particles are also contemplated. Larger particles may be utilized to house organic matter, nutrients, sugars, starches, photo protectants, tackifiers, or a combination thereof.

The particles may have a uniform particle size. Alternatively, a bulk of the porous particles in a single batch may include variety of sizes. For example, majority of the porous particles may have a first size while the remainder of the particles may have at least a second size, alternatively a third, fourth, fifth size, etc.

When the particles are inoculated, the particles may be stored in a dry place with temperatures of about −20° C. (−4° F.) to about 50° C. (122° F.). When desired, the particles may be applied to the remedial site in batches. A non-limiting example batch may weight about 25 lbs (11.4 kg) to 300 lbs (136 kg), 50 lbs (22.7 kg) to 200 lbs (90.7 kg), or 75 lbs (34 kg) to 100 lbs (45.4 kg). Spreading of the batches may be done, for example, aerially, hydraulically, via agricultural equipment such as spreaders, or otherwise. Once applied, the inoculated particles are designed to remain at the remedial site until sufficient moisture content activates the biological portion and or longer. The activation may occur once natural precipitation ensues in sufficient amount. Alternatively, watering initiated post-application may activate the organisms such that the remediation is initiated.

Concentration of cyanobacteria in a dry or liquid inoculant may be about 10 to 90, 20 to 80, or 30 to 70%. Concentration of cyanobacteria in an inoculant may be about, at least about, or up to 1, 2, 3, 4, 5, 6, 7, 8, 9, 10, 11, 12, 13, 14, 15, 16, 17, 18, 19, 20, 22, 24, 26, 28, 30, 32, 34,3 36, 38, 40, 42, 44, 46, 48, 50, 55, 60, 65, 70, 75, 80, 85, 90, 95, or 99%.

The initial amount/dehumidified amount of inoculant or biological portion on the particles may be about, at least about, or up to about 500 to 40,000 g of inoculant or biological portion acre of particles. The initial amount/dehumidified amount of inoculant or biological portion on the particles may be about, at least about, or up to about 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 g of inoculant or biological portion/acre of particles.

Application density of the particles onto the soil at an application site may be about 10 to 5000, 100 to 2500, or 5000 to 2000 lbs/acre. Application density of the particles onto the soil at an application site may be about, up to about, or at least about 10, 20, 30, 40, 50, 60, 70, 80, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1050, 1100, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3450, 3400, 3500, 3550, 3600. 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800,4850, 4900, 4950, or 5000 lbs/acre.

Application density of a liquid inoculant at an application site may be about, at least about, or up to about 0.5 to 40, 5 to 30, or 10 to 20 gal/acre. Application density of a liquid inoculant at an application site may be about, at least about, or up to about 0.5, 1, 2, 2.5, 3, 3.5, 4, 4.5, 5, 5.5,6. 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, or 40 gal/acre.

When applied hydraulically. a mixture including the inoculant, inoculated particles, bare particles with no inoculant, growing medium or mulch, a cyanobacteria food source. macronutrients, micronutrients, tackifier, bio stimulants, plant hormones, the like, or their combination may be mixed with water. The amount of water may vary depending on water availability at the remediation site and requirements of a specific hydroseeding application. An example amount of water may be about 1 to 70, 10 to 50, or 20 to 30 vol %, based on the total volume of the mixture. Example amount of water may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 46, 48, 50, 52, 54, 56, 58, 60, 62, 64, 66, 68, or 70 vol %, based on the total volume of the mixture.

Application density of additional components present within the mixture such as food source, tackifiers, bio stimulants, and the like may be in the amount of about, up to about, or at least about 0.1 to 25, 5 to 20, or 10 to 15 gal/acre or lbs/acre. Application density of additional components present within the mixture such as food source, tackifiers, bio stimulants, and the like may be in the amount of about, up to about, or at least about 0.1, 0.2, 0.3, 0.4, 0.5, 0.6, 0.7, 0.8, 0.9, 1, 1.1, 1.2, 1.4, 1.6, 1.8, 2, 2.2, 2.4, 2.6, 2.8, 3, 3.2, 3.4, 3.6, 3.8, 4, 4.2, 4.4, 4.6, 5, 5.2, 5.4, 5.6, 5.8, 6, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, 10, 10.5, 11, 11.5, 12, 12.5, 13, 13.5, 14, 14.5, 15, 15.5, 16, 16.5, 17, 17.5, 18, 18.5, 19, 19.5, 20, 21, 22, 23, 24, or 25 gal/acre or lbs/acre.

### EXAMPLES

First Trial—Growth Chamber trial—Examples 1-40

Preparation of the Inoculated Particles

Inoculant 1 was prepared by adding the following cyanobacteria species: Microcoleus and Nostoc in an amount of 50/50 ratio into the respective cyanobacteria growing broth.

Inoculant 2 was prepared by adding the following cyanobacteria species: Microcoleus and Nostoc in an amount of 50/50 ratio into a nutrient broth.

20 ml of Inoculant 1 or Inoculant 2 was applied to a mass of untreated ceramic particles according to Table 3 below, yielding an average of 9.0 g of increased mass/0.5 lb (0.23 kg) of treated particles. 226.8 g equals ½ pound, which translates to about 18.0 g/lbs (39.68 g/kg). Coating of the particles was performed using a mixer that allows mixing of the particles while the inoculant was being applied onto the particles. To apply the coating, the Inoculants 1 and 2 were diluted and blended to become less viscous.

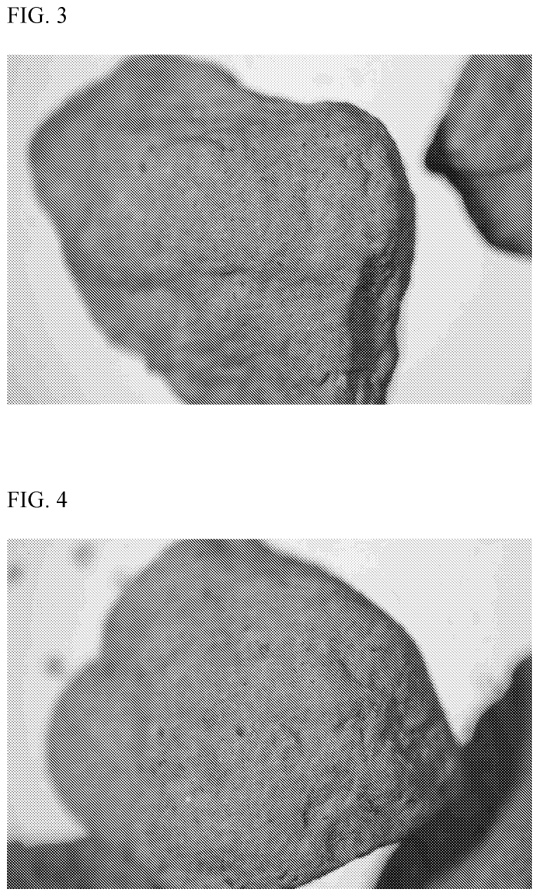

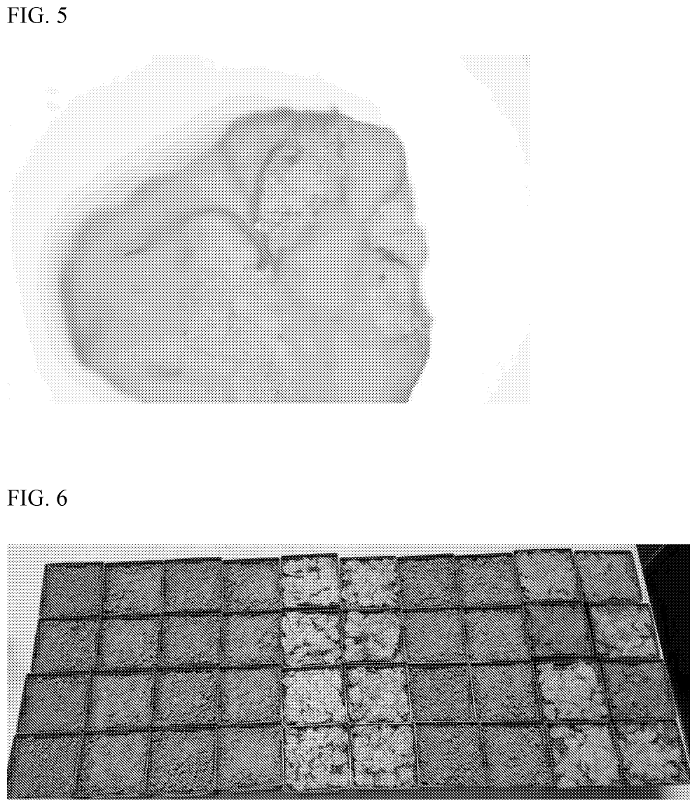

An example uncoated porous ceramic particle can be seen in FIG. 3. An example porous ceramic particle coated with Inoculant 1 is depicted in FIG. 4. FIG. 5 depicts a coated porous ceramic particle showing cyanobacteria fibers attached to the particle surface.

TABLE 3
Coating mass added to the particles

| Particle | Untreated | Treated | Mass added |
|---|---|---|---|
| size [μm] | Mass [g] | Mass [g] | by coating [g] |
| 24 × 48 | 226.8 | 235.0 | 8.2 |
| 5 × 30 | 226.8 | 234.3 | 7.5 |
| 8 × 16 | 226.8 | 234.3 | 7.5 |
| ⅜″ × # 8 Pan | 226.8 | 239.6 | 12.8 |
| Average | 226.8 | 235.8 | 9.0 |

Concentration of the Inoculant 1 or 2 varied from 10% to 90%, as can be seen in Table 4 below.

TABLE 4
The concentration of the Inoculant 1 or
Inoculant 2 as applied onto the particles.

| Inoculant 1 or 2 | Application density | Outcome | |
|---|---|---|---|
| concentration [%] | [lbs/acre of particles] | [g of Inoculant/acre of particles] | |
| 10 | 500 | 500*18*0.1 = | 900 |
| 25 | 500 | 500*18*0.25 = | 2,250 |
| 50 | 500 | 500*18*0.5 = | 4,500 |
| 75 | 500 | 500*18*0.75 = | 6,750 |
| 90 | 500 | 500*18*0.9 = | 8,100 |
| 10 | 2,000 | 2,000*18*0.1 = | 3,600 |
| 25 | 2,000 | 2,000*18*0.25 = | 9,000 |
| 50 | 2,000 | 2,000*18*0.5 = | 18,000 |
| 75 | 2,000 | 2,000*18*0.75 = | 27,000 |
| 90 | 2,000 | 2000*18*0.9 = | 32,400 |

The coated particles were placed in a flask and water was added to the coated particles to reach volume of 150 mL. From 50-100% of the applied Inoculum 1 or Inoculum 2 was retained on the particles, with average retention of 75%.

TABLE 5
Decrease in volume due to coating

| | | Untreated | Treated | Decrease in |
|---|---|---|---|---|
| Particle | | Volume | Volume | Volume due to |
| size [μm] | | [mL] | [mL] | coating [mL] |
| 24 × | 48 | 117/150 = 0.785 | 97/150 = 0.65 | 20 |
| 5 × | 30 | 105/150 = 0.70 | 93/150 = 0.62 | 12 |
| 8 × | 16 | 102/150 = 0.68 | 86/150 = 0.57 | 16 |
| ⅜″ × | # 8 Pan | 90/150 = 0.60 | 80/150 = 0.53 | 10 |

| | | | |
|---|---|---|---|
| Average | 104/150 = 0.96 | 89/150 = 0.59 | 15 |

Preparation of Soil Samples

40 examples were prepared. Each example was contained within a pot having measurements of 4×4 inches, thus 16 inch² or 0.1111 ft². Each pot included native Arizona desert soil. Particles inoculated with Inoculant 1 or Inoculant 2 were applied by hand with pouring tray to simulate spreading in the field on top of 36 samples as described in Tables 6 and 7. Samples 1-4 were control examples with no application of particles. Examples 1-40, as prepared, arc depicted in FIG. 6.

TABLE 6
Types of treatment applied to Examples 1-20.

| | Example no. | | | | |
|---|---|---|---|---|---|
| | 1-4 | 5-8 | 9-12 | 13-16 | 17-20 |
| Treatment | Control | Uncoated | Coated | Coated | Coated |
| | with no | particles | particles- | particles- | particles- |
| | treat- | | inoculant 1 | inoculant 1 | inoculant 1 |
| | ment | | | | |
| Density of | — | 1250 | 500 | 2,000 | 500 |
| application | | | | | |
| [lb/acre] | | | | | |
| Type of | — | Dry | Dry | Dry | Wet with |
| application | | | | | mulch carrier |
| Particle | — | 24 × 48 | 24 × 48 | 24 × 48 | 24 × 48 |
| size | | | | | |
| Additional | — | — | — | — | (1) 2,000 |
| treatment | | | | | lbs/acre- |
| | | | | | undyed |
| | | | | | mulch* |
| | | | | | (2) 20 lbs/ |
| | | | | | acre |
| | | | | | biochar |

*mulch used was Conwed Fibers ® Hydro Mulch ® 2,000

TABLE 7
Types of treatmeat applied to Examples 21-40.

| | Sample no. | | | | |
|---|---|---|---|---|---|
| | 21-24 | 25-28 | 29-32 | 33-36 | 37-40 |
| Treatment | Coated | Coated | Coated | Coated | Coated |
| | particles- | particles- | particles- | particles- | particles- |
| | inoculant 1 | inoculant 2 | inoculant 2 | inoculant 2 | inoculant 2 |
| Density of | 2,000 | 500 | 2,000 | 500 | 2,000 |
| application | | | | | |
| [lbs/acre] | | | | | |
| Type of | Wet with | Dry | Dry | Wet with | Wet with |
| application | mulch | | | mulch | mulch |
| | carrier | | | carrier | carrier |
| Particle | 24 × 48 | 24 × 48 | 24 × 48 | 24 × 48 | 24 × 48 |
| size | | | | | |
| Additional | (1) 2,000 | — | — | (1) 2,000 | (1) 2,000 |
| treatment | lbs/acre- | | | lbs/acre- | lbs/acre- |
| | undyed | | | undyed | undyed |
| | mulch* | | | mulch* | mulch* |
| | (2) 20 | | | (2) 20 | (2) 20 |
| | lbs/acre | | | lbs/acre | lbs/acre |
| | biochar | | | biochar | biochar |

*mulch used was Conwed Fibers ® Hydro Mulch ® 2,000

TABLE 8
Unit conversion of application density

| Density of | Density of | Density of | Density of |
|---|---|---|---|
| inoculated | inoculated | inoculated | inoculated |
| particles | particles | particles | particles |
| [lb/acre] | [lb/ft²] | [g/ft²] | [g/pot] |
| 500 | 0.011478 | 5.21 | 0.5788 |
| 1,250 | 0.028696 | 13.0 | 1.444 |
| 2,000 | 0.045914 | 20.83 | 2.314 |
| 20 | 0.000459 | 0.21 | 0.231 |

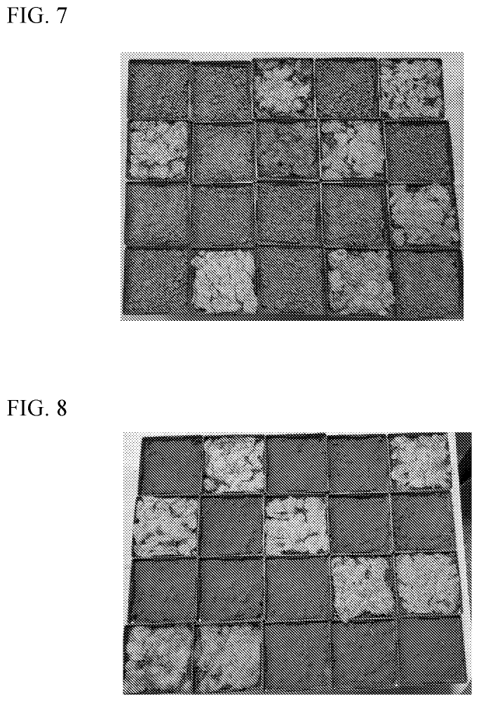

Water in the amount of 62 sprays equivalent to 0.2 inches or 52 mL was applied to each sample. All samples were then placed into a Thermo Scientific Diurnal Growth Chamber, model #FFU2064DW8, Serial #WB72043709. 20 samples were placed on the top shelf, 20 samples were placed on the bottom shelf of the Chamber. Samples on both shelves were randomized, as can be seen in FIGS. 7 and 8. FIG. 7 shows randomized samples on the top shelf of the Chamber before water application. FIG. 8 shows randomized samples on the bottom shelf of the Chamber after water application. Additional water in the amount of 62 sprays equivalent to 0.2 inches or 52 mL was applied to each sample every week of the trial.

All samples were exposed to 12 hours of light, luminous emittance of about 20,000 Lux, simulating daytime hours, in the temperature of about 90° F. (32.22° C.)±10° F. (26.67° C. to 37.78° C.) and 12 hours of darkness, simulating nighttime hours, in the temperature of about 65° F. (18.33° C.)±10° F. (12.78° C. to 23.89° C.). Relative humidity varied, but was less than about 50%.

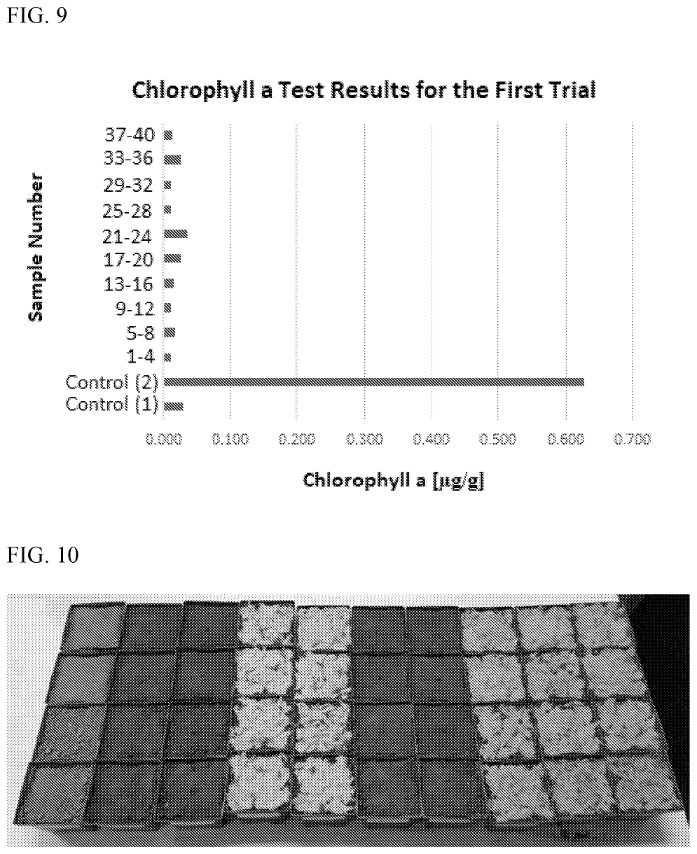

Examples 1-40 were tested 8 weeks after installation. In addition, two Control Samples were added for comparison: (1) a soil sample from an Arizona field and (2) particles having a size of 24×48 μm were coated and kept in a dark cabinet at ambient temperature for 14 days prior to testing as a positive control to assess viability of the Inoculants 1 and 2 in packaging. Testing of examples 1-40 and Controls (1) and (2) was done by measuring Chlorophyll a content of each sample, which is indicative of the cyanobacteria growth within the sample. The measurements were taken according to Chlorophyll a phaeopigment analysis of sediment/soil, EPA Method 445 for analysis of extracts. Equipment used was Turner Model TD700 Fuorometer. multi-optional raw fluorescence mode. The results are captured in Table 9 below and in FIG. 9.

TABLE 9
Results of the First Trial

| Example No. | Treatment | Chlorophyll a [μg/g] |
|---|---|---|
| Control (1) | Field Soil-AZ | 0.030 |
| Control (2) | 24 × 48 Coated | 0.628 |
| 1-4 | Control Soil-AZ | 0.012 |
| 5-8 | Control w/Uncoated 24 × 48 | 0.017 |
| 9-12 | Inoculant 1, Dry 500 lb/acre | 0.011 |
| 13-16 | Inoculant 1, Dry 2,000 lb/acre | 0.016 |
| 17-20 | Inoculant 1, Wet 500 lb/acre | 0.025 |
| 21-24 | Inoculant 1, Wet 2,000 lb/acre | 0.036 |
| 25-28 | Inoculant 1, Dry 500 lb/acre | 0.012 |
| 29-32 | Inoculant 2, Dry 2,000 lb/acre | 0.012 |
| 33-36 | Inoculant 2, Wet 500 lb/acre | 0.026 |
| 37-40 | Inoculant 2, Wet 2,000 lb/acre | 0.015 |

Second Trial—Growth Chamber Trial—Examples 40-80

Preparation of the Inoculants

Inoculant 3 was prepared by adding the following cyanobacteria species: Microcoleus and Nostoc in an amount of 50/50 ratio into the respective cyanobacteria growing broth.

Inoculant 4 was prepared by adding the following cyanobacteria species: Microcoleus and Nostoc in an amount of 50/50 ratio into a nutrient broth.

Unlike in the First Trial, in the Second Trial, the Inoculants 3 and 4 were not coated onto the particles, but rather the Inoculants 3 and 4 were applied directly to the soil samples as a liquid inoculant.

Preparation of Soil Samples

40 examples were prepared. Each example was contained within a pot having measurements of 4×4 inches, thus 16 inch² or 0.1111 ft². Each pot included native Arizona desert soil. Liquid Inoculant 3 or Inoculant 4 were applied by hand with pouring tray to simulate spreading in the field on top of 36 samples as described in Tables 10 and 11. Examples 41-44 were control samples of field Arizona soil with no application of the disclosed particles. Examples 41-80, as prepared, are depicted in FIG. 10.

TABLE 10
Types of treatment applied to examples 41-60 containing Inoculant 3.

| | Example no. | | | | |
|---|---|---|---|---|---|
| | 41-44 | 45-48 | 49-52 | 53-56 | 57-60 |
| Density of | Control | 5 | 10 | 5 | 10 |
| application | with no | | | | |
| [gal/acre] | treatment | | | | |
| Type of | — | wet | wet | wet with | wet with |
| application | | | | mulch | mulch |
| | | | | carrier | carrier |
| Additional | — | — | — | (1) 2,000 lbs/ | (1) 2000, lbs/ |
| treatment | | | | acre-undyed | acre-undyed |
| | | | | mulch* | mulch* |
| | | | | (2) 20 lbs/acre | (2) 20 |
| | | | | guar food | lbs/acre |
| | | | | source | biochar |

*mulch used was Conwed Fibers ® Hydro Mulch ® 2,000

TABLE 11
Types of treatment applied to samples 61-80 containing Inoculant 4.

| | Example no. | | | | |
|---|---|---|---|---|---|
| | 61-64 | 65-68 | 69-72 | 73-76 | 77-80 |
| Density of | 5 | 10 | 5 | 10 | 10-mixed into |
| application | | | | | the top layer of |
| [gal/acre] | | | | | soil |
| Type of | wet | wet | wet with mulch | wet with mulch | wet with mulch |
| application | | | carrier | carrier | carrier |
| Additional | — | — | (1) 2,000 lbs/acre- | (1) 2,000 lbs/acre- | (1) 2,000 lbs/acre- |
| treatment | | | undyed mulch* | undyed mulch* | undyed mulch* |
| | | | (2) 20 lbs/acre | (2) 20 lbs/acre | (2) 20 lbs/acre |
| | | | guar food source | guar food source | guar food source |

*mulch used was Conwed Fibers ® Hydro Mulch ® 2,000

Water in the amount of 62 sprays equivalent to 0.2 inches or 52 mL was applied to each sample. All samples were then placed into a Thermo Scientific Diurnal Growth Chamber, model #FFU2064DW8, Serial #WB72043709. 20 samples were placed on the top shelf, 20 samples were placed on the bottom shelf of the Chamber. Samples on both shelves were randomized. Additional water in the amount of 62 sprays equivalent to 0.2 inches or 52 mL was applied to each sample every week of the trial.

All samples were exposed to 12 hours of light, luminous emittance of about 20,000 Lux, simulating daytime hours, in the temperature of about 90° F. (32.22° C.)±10° F. (26.67° C. to 37.78° C.) and 12 hours of darkness, simulating nighttime hours, in the temperature of about 65° F. (18.33° C.)±10° F. (12.78° C. to 23.89° C.). Relative humidity varied, but was less than about 50%.

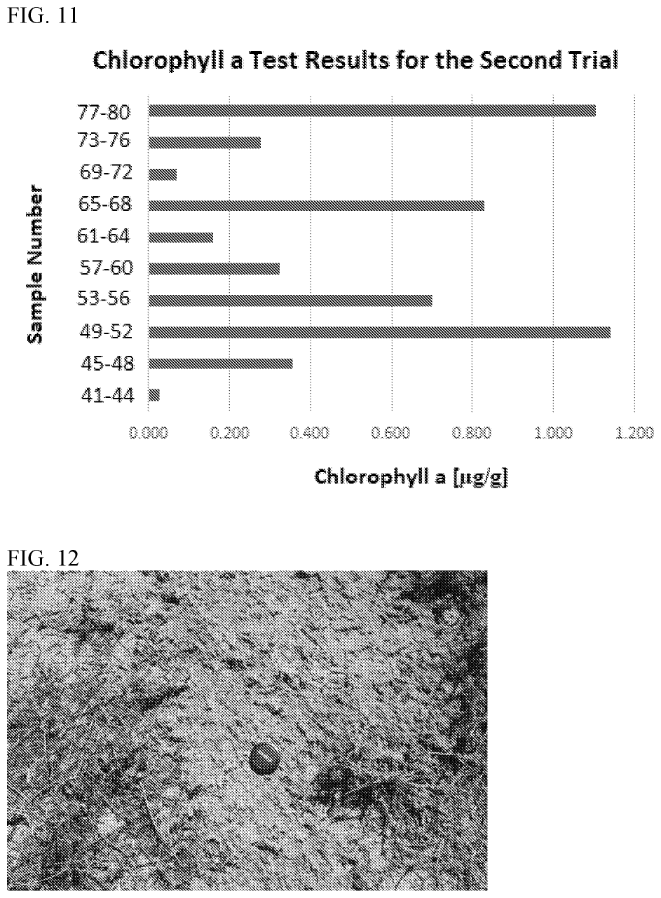

Samples 41-80 were tested 4 weeks after installation. Testing of samples 41-80 was done by measuring Chlorophyll a content of each example, which is indicative of the cyanobacteria growth within the sample. The measurements were taken according to Chlorophyll a phaeopigment analysis of sediment/soil, EPA Method 445 for analysis of extracts. Equipment used was Turner Model TD700 Fluorometer, multi-optional raw fluorescence mode. The results are captured in Table 12 below and in FIG. 11.

TABLE 12
Results of the Second Trial

| Sample No. | Treatment | Chlorophyll a [μg/g] |
|---|---|---|
| 41-44-Control | Field Soil-AZ | 0.029 |
| 45-48 | Inoculant 3, liquid, 5 gal/acre | 0.358 |
| 49-52 | Inoculant 3, liquid, 10 gal/acre | 1.140 |
| 53-56 | Inoculant 3, liquid, 5 gal/acre | 0.699 |
| | with mulch | |
| 57-60 | Inoculant 3, liquid, 10 gal/acre | 0.325 |
| | with mulch | |
| 61-64 | Inoculant 4, liquid, 5 gal/acre | 0.158 |
| 65-68 | Inoculant 4, liquid, 10 gal/acre | 0.828 |
| 69-72 | Inoculant 4, liquid, 5 gal/acre | 0.070 |
| | with mulch | |
| 73-76 | Inoculant 4, liquid, 10 gal/acre | 0.276 |
| | with mulch | |
| 77-80 | Inoculant 4, liquid, 10 gal/acre | 1.103 |
| | soil mix mulch | |

Third Trial—Field Trial—Examples 40-80

Preparation of the Inoculants

Inoculant 5 was prepared by adding the following cyanobacteria species: Nostoc in an amount of 50/50 ratio into the respective cyanobacteria growing broth.

Inoculant 6 was prepared by adding the following cyanobacteria species: Microcoleus in an amount of 50/50 ratio into the respective cyanobacteria growing broth.

Inoculant 7 was prepared by adding the following cyanobacteria species: Microcoleus and Nostoc in an amount of 50/50 ratio into the respective cyanobacteria growing broth.

Field Conditions

The Third Trial was conducted on a plot in Southwest U.S.A. in arid/semi-arid climatic conditions. The site is a retired farm land, site conditions are typical of other areas in the region. Project elevation was about 3,300 feet (1,005.84 m); average annual rainfall is about 9.7 inches/year (24.638 cm/year); the average expected rainfall during months of install was about 1.5 incites (3.81 cm). Climatic conditions for the site are listed below in Table 13.

TABLE 13
Average climate conditions for the test site of the Third Trial

| | January | February | March | April | May | June | July | August | September | October | November | December | Average |
|---|---|---|---|---|---|---|---|---|---|---|---|---|---|
| Average | 61 | 65 | 72 | 80 | 90 | 98 | 98 | 86 | 92 | 83 | 70 | 60 | 80.4 |
| high [° F.] | | | | | | | | | | | | | |
| Average | 29 | 33 | 38 | 45 | 53 | 62 | 68 | 67 | 60 | 48 | 36 | 29 | 47.3 |
| low [° F.] | | | | | | | | | | | | | |
| Average | 0.75 | 0.75 | 0.63 | 0.28 | 0.24 | 0.28 | 1.5 | 1.93 | 1.02 | 0.87 | 0.55 | 0.87 | 0.8 |
| precipitation | | | | | | | | | | | | | |
| [inch] | | | | | | | | | | | | | |

The site of about 2 acres (4,046.86 m²) was fenced and subdivided into 8 plots, each measuring about 50×50 feet (15.24×15.24 m). The slope gradient of the site was flat, slope length was about 364 feet (110.98 m) and slope width about 232 feet (70.73 m).

Site Preparation

No surface preparation was done prior to the Third Trial such that the Trial used existing conditions. Irrigation was not used before or during the Third Trial. A pre-test soil analysis revealed a phosphorus deficiency, phosphorus fertilizer 0-46-0 at 16.7 lb/acre (18.7 kg/ha) was used on the site to improve the phosphorus content as part of the treatment. No additional soil amendments were made.

Test Plots

10 test plots were prepared within the testing site. Each testing plot measured about 50×50 feet (15.24×15.24 m) with the exception of Example 88, which was applied to a plot having a size of about 12.5×50 feet (3.81×15.24 m). Tables 14 and 15 show Examples 81-90. As can be seen below, guar was used as a food source and tackifier for mulch fiber. Mulch or wood fiber was used as protective layer on the liquid applications to manage erosion control. Seaweed extract was used as a bio stimulant. Phosee broth relates to a phosphorus-based liquid culture medium with metabolites.

TABLE 14
Types of treatment applied to examples 81-85.

| | Example no. | | | | |
|---|---|---|---|---|---|
| | 81 | 82 | 83 | 84 | 85 |
| Inoculant(s) | Control | 5, 6 | 5, 6 | 7 | 5, 6, 7 |
| | with no | | | | |
| | treatment | | | | |
| Density of | — | each at 10 | each at 20 | 20 | each at 10 |
| application | | | | | |
| [gal/acre] | | | | | |
| Type of | — | liquid | liquid | liquid | liquid |
| application | | | | | |
| Additional | — | 10 gal/acre | 20 gal/acre | Mulch cap | 10 gal/acre |
| treatment | | Phosee broth | Phosee broth | including: | Phosee broth |
| | | Mulch cap | Mulch cap | 2,000 lbs/ | Mulch cap |
| | | including: 2,000 | including: 2,000 | acre-undyed | including: 2,000 |
| | | lbs/acre-undyed | lbs/acre-undyed | mulch*, guar | lbs/acre- |
| | | mulch*, | mulch*, | food source, | undyed mulch*, |
| | | guar food | guar food | seaweed | guar food |
| | | source, seaweed | source, seaweed | extract, | source, seaweed |
| | | extract, | extract, | phosphorus | extract, |
| | | phosphorus | phosphorus | | phosphorus |

*mulch used was HydraFiber ®

TABLE 15
Types of treatment applied to examples 86-90.

| | Example no | | | | |
|---|---|---|---|---|---|
| | 86 | 87 | 88 | 89 | 90 |
| Inoculant(s) | 5, 6 | 7 | 6 | 5, 6, 7 | 5, 6, 7 |
| Density of | each at 10 | 20 | 20 | each at 10 | 32, 32, 37 |
| application | | | | | respectively |
| [gal/acre] | | | | | |
| Type of | liquid- | liquid- | dry-coated | liquid- | liquid- |
| application | blended in | blended in | particles at | blended in | blended in tank |
| | tank with | tank with | 2,000 lbs/acre | tank with | with mulch |
| | mulch | mulch | | mulch | |
| Additional | (1) 10 gal/acre | (1) 2,000 | (1) 2,000 | (1) 10 gal/acre | (1) 2,000 |
| treatment | Phosee broth | lbs/acre- | lbs/acre- | Phosee broth | lbs/acre- |
| | (2) 2,000 | undyed | undyed mulch* | (2) 2,000 | undyed |
| | lbs/acre- | mulch* = | (2) guar food | lbs/acre- | mulch* = 2 bales |
| | undyed | 2.5 bales | source | undyed | (3) 2 lbs guar |
| | mulch* = | (2) guar | (3) seaweed | mulch* = 2 | food source |
| | 2.5 bales | food source | extract | bales | (4) seaweed |
| | (3) guar | (3) seaweed | (4) | (3) guar food | extract |
| | food source | extract | phosphorus | source | (5) |
| | (4) seaweed | (4) | | (4) seaweed | phosphorus |
| | extract | phosphorus | | extract | |
| | (5) | | | (5) | |
| | phosphorus | | | phosphorus | |

*mulch used was HydraFiber ®

Application rates for Examples 81-90 were as follows unless otherwise specified in Tables 14 and 15:

Inoculant, phosee broth at 10 gal/acre*0.057 acres=0.57 gal (2.16 L), 2,160 mL;

Inoculant, phosee broth at 20 gal/acre*0.05 acres=1.15 gal (4.35 L), 4,360 mL;

Mulch & coated particles: 2,000 lb/acre*0.057 acre=115 lb, at ¼ plot=28.75 lb (13.04 kg);

Guar: 100 gal/acre*0.057 acres=5.7 lb (2.59 kg):

Seaweed extract: 2 g/gal, 228 gal and water=456 g/plot=1 lb (0.45 kg); and

Phosphorus: 16.67 lb/acre*0.057=0.95 lbs˜1 lbs (0.45 kg).

The liquid examples 82-87, 89, and 90 were applied by a hydroseeder, Brand Bowie, Model 1100 with a truck-mount configuration and mechanical agitation, gear pump type, and liquid capacity of about 1,000 gallon (3,785.41 L). Bach hydroseeder tank included about 250 gal (946.35 L) of water, the inoculant(s) and or other components as listed in Tables 14 and 15 above. FIG. 12 shows an example of a finished plot for Examples 82-85. The mulch cap was applied over examples 82-85 from the same hydroseeder to form the protective mulch cap. In contrast, in Examples 86, 87, 89, and 90, the mulch was mixed with the liquid inoculant in the hydroseeder tank.

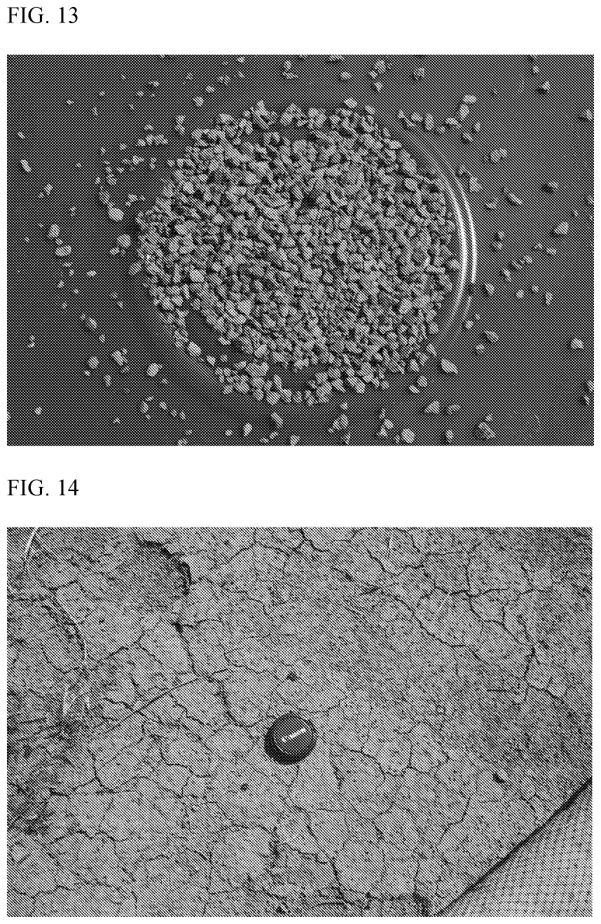

The dry example 88 was prepared by coating ceramic particles disclosed herein with Inoculant 6 and growing the cyanobacteria on the particles such that the particles may serve as a physical support for the organisms. The cyanobacteria inoculated particles were harvested, allowed to dry to a moisture content of about 15-20%, and spread by hand onto the respective plot. FIGS. 13 and 14 show the inoculated particles before and after application onto the respective plot.

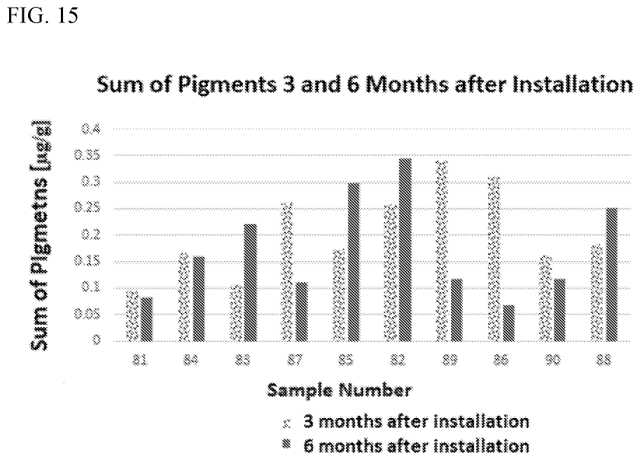

Examples 81-90 were tested 3 months and 6 months after installation. Testing of Examples 81-90 was done by measuring Chlorophyll a, Phaeopigment content, and sum of pigments of each sample, which is indicative of the cyanobacteria growth within the sample. The measurements were taken according to Chlorophyll a phaeopigment analysis of sediment/soil. EPA Method 445 for analysis of extracts. Equipment used was Turner Model TD700 Fluorometer. multi-optional raw-fluorescence mode. The results are captured in Tables 16 and 17 below and in FIG. 15.

TABLE 16
Results of the Third Trial three months after installation

| | Chloro- | Phaeo- | Sum of | Sum |
|---|---|---|---|---|
| Example | phyll a | pigment | pigments | Pigments |
| No. | [μg/g] | [μg/g] | [μg/g] | vs. Control |
| 81-control | 0.042 | 0.051 | 0.093 | — |
| 82 | 0.102 | 0.157 | 0.259 | 2.78 |
| 83 | 0.041 | 0.065 | 0.106 | 1.14 |
| 84 | 0.076 | 0.091 | 0.167 | 1.80 |
| 85 | 0.071 | 0.102 | 0.173 | 1.86 |
| 86 | 0.138 | 0.171 | 0.309 | 3.32 |
| 87 | 0.130 | 0.130 | 0.260 | 2.80 |
| 88-inoculated | 0.077 | 0.106 | 0.183 | 1.97 |
| particles | | | | |
| 89 | 0.141 | 0.199 | 0.34 | 3.66 |
| 90 | 0.049 | 0.112 | 0.161 | 1.73 |

TABLE 17
Results of the Third Trial 6 months after installation

| | Chloro- | Phaeo- | Sum of | Sum |
|---|---|---|---|---|
| Example | phyll a | pigment | pigments | Pigments |
| No. | [μg/g] | [μg/g] | [μg/g] | vs. Control |
| 81-control | 0.028 | 0.055 | 0.081 | — |
| 82 | 0.152 | 0.192 | 0.344 | 4.16 |
| 83 | 0.082 | 0.138 | 0.220 | 2.66 |
| 84 | 0.065 | 0.095 | 0.160 | 1.94 |
| 85 | 0.124 | 0.173 | 0.297 | 3.59 |
| 86 | 0.021 | 0.048 | 0.069 | 0.83 |
| 87 | 0.035 | 0.076 | 0.112 | 1.35 |
| 88- | 0.105 | 0.145 | 0.250 | 3.03 |
| inoculated | | | | |
| particles | | | | |
| 89 | 0.035 | 0.081 | 0.116 | 1.41 |
| 90 | 0.040 | 0.076 | 0.117 | 1.41 |

While exemplary embodiments are described above, it is not intended that these embodiments describe all possible forms of the disclosure. Rather, the words used in the specification are words of description rather than limitation, and it is understood that various changes may be made without departing from the spirit and scope of the disclosure. Additionally, the features of various implementing embodiments may be combined to form further embodiments of the disclosure.

What is claimed is:

1. A soil restoration system comprising:
a plurality of dry particles, each particle inoculated with dehumidified biological material including at least one species of cyanobacteria, the cyanobacteria being physically supported by the particles, and the cyanobacteria being activatable by a threshold amount of moisture so that when the cyanobacteria is activated at an application site, the cyanobacteria multiplies to form a biological soil crust capable of facilitating plant seed catchment and vascular plant germination at the application site.

2. The system of claim 1, wherein the particles are ceramic particles.

3. The system of claim 1, wherein the cyanobacteria comprise Microcoleus genera, Nostoc genera, of their combination.

4. The system of claim 1, w herein the system further comprises at least one of a cyanobacteria food source, macronutrients, micronutrients, tackifier, bio stimulants, or plant hormones.

5. The system of claim 1, wherein the biological material further comprises at least one species of fungi.

6. The system of claim 1, wherein the particle total porosity is about 50 to 95 volume %.

7. The system of claim 1, wherein application density of the plurality of particles on the application site is about 500 to 2000 lbs/acre.

8. The system of claim 1, wherein the particles have a concentration of the dehumidified biological material of about 500 to 40,000 g/acre of particles.

9. A soil restoration system comprising:
a plurality of dry particles, each particle inoculated with dehumidified biological material including at least one species of cyanobacteria, the cyanobacteria being physically supported by the particles, and the cyanobacteria being activatable by a threshold amount of moisture so that when the cyanobacteria is activated at an application site, the cyanobacteria multiplies to form a biological soil crust capable of facilitating plant seed catchment and vascular plant germination at the application site; and
growing medium or mulch having a density of about 60 kg/m³ or lower.

10. The system of claim 9, wherein the plurality of particles and the growing medium form a mixture.

11. The system of claim 9, wherein the growing medium forms a protective layer over the plurality of particles.

12. The system of claim 9, wherein the system further comprises at least one of a cyanobacteria food source, macronutrients, micronutrients, tackifier, bio stimulants, or plant hormones.

13. The system of claim 9, wherein the biological material further comprises at least one species of fungi.

14. The system of claim 9, wherein the biological portion comprises bacterial genera matching a separate biological material to be present at the application site.

15. The system of claim 9, wherein application density of the plurality of particles on the application site is about 500 to 2000 lbs/acre.

16. A soil restoration system comprising:
a liquid inoculant comprising at least one species of cyanobacteria to be applied at an application site such that when the cyanobacteria is activated at an application site, the cyanobacteria multiplies to form a biological soil crust capable of facilitating plant seed catchment and vascular plant germination at the application site; and
growing medium or mulch having a density of about 60 kg/m³ or lower.

17. The system of claim 16, wherein the liquid inoculant and the growing medium form a mixture.

18. The system of claim 16, wherein the growing medium forms a protective layer over the plurality of particles.

19. The system of claim 16, wherein the system further comprises at least one of a cyanobacteria food source, macronutrients, micronutrients, tackifier, bio stimulants, or plant hormones.

20. The system of claim 16, wherein application density of the liquid inoculant on the application site is about 5 to 20 gal/acre.
